(19) United States
(12) Patent Application Publication
(10) Pub. No.: US 2018/0250403 A1
(43) Pub. Date: Sep. 6, 2018

(54) CONJUGATES

(71) Applicant: AGENCY FOR SCIENCE, TECHNOLOGY AND RESEARCH, Singapore (SG)

(72) Inventors: Paul Lorenz BIGLIARDI, Singapore (SG); Mei BIGLIARDI-QI, Singapore (SG); Bhimsen ROUT, Singapore (SG); Brendan BURKETT, Singapore (SG); Aakanksha PANT, Singapore (SG)

(21) Appl. No.: 15/754,573

(22) PCT Filed: Aug. 24, 2016
(86) PCT No.: PCT/SG2016/050409

§ 371 Date: Feb. 22, 2018

(30) Foreign Application Priority Data
Aug. 24, 2015 (SG) 10201506686W

(51) Int. Cl. A61K 41/00; A61K 47/10; A61K 47/18; A61P 35/00

(52) U.S. Cl. CPC A61K 41/0038; A61K 47/10; G01N 33/57484; A61P 35/00; A61K 47/18

(57) ABSTRACT

The present invention relates to improved compositions for photodynamic therapy (PDT) for the selective destruction of malignant, diseased, or infected cells or infective agents without causing damage to normal cells. In an embodiment, the composition comprises a photosensitising agent coupled to a ligand, wherein the ligand selectively binds to a targeted receptor and comprises an isolated peptide molecule having less than 10 or 8 amino acids.

The present invention relates to improved compositions for photodynamic therapy (PDT) for the selective destruction of malignant, diseased, or infected cells or infective agents and change of the pigment of the cells with minimum or no collateral damage to normal cells.

PDT is a minimally invasive treatment for a range of conditions where diseased cells and tissues need to be removed. Unlike ionising radiation, it can be administered repeatedly at the same site. Its use in cancer treatment is attractive because the use of conventional modalities such as chemotherapy, radiotherapy or surgery does not preclude the use of PDT and vice versa. PDT is also finding other applications where specific cell populations must be destroyed, such as blood vessels (in age-related macular degeneration or in cancer), the treatment of immune disorders, cardiovascular disease, and microbial infections.

PDT is a two-step or binary process starting with the administration of the photosensitizer agent or drug, by intravenous injection, or topical application for skin disorder. The physico-chemical nature of the drug causes it to be preferentially taken up by cancerous cells or other target cells. Once a favourable drug uptake ratio between tumour (or other target) tissues versus normal tissues is obtained, the second step is the activation of the agent or drug with a specific dose of light, at a particular wavelength. The photosensitizer, in its ground or singlet state absorbs a photon of light at a specific wavelength. This results in a short-lived excited singlet state. This can be converted by intersystem crossing to a longer-lived triplet state. It is this form of the sensitizer which carries out various cytotoxic actions.

The main classes of reactions are photooxidation by radicals (type I reaction), photooxidation by singlet oxygen (type II reaction), and photoreaction not involving oxygen (type III reaction). The triplet state form of the photosensitizer causes the conversion of molecular oxygen found in the cellular environment into reactive oxygen species (ROS) primarily singlet oxygen (O₂) via a Type II reaction. If an activated photosensitizer interacts with cellular components, a Type I reaction occurs where electrons or protons are abstracted forming radicals such as hydroxyl radicals.

These molecular species cause damage to cellular components such as DNA, proteins and lipids. A Type III mechanism has also been proposed where the triplet state photosensitizer interacts with free radicals to cause cellular damage. The site of cellular damage depends upon the type of photosensitizer, duration of incubation, type of cells and mode of delivery. Hydrophobic photosensitizers tend to damage cell membranes, whereas cationic photosensitizers localise within membrane vesicles such as mitochondria and cause damage there.

The light activation of ROS is highly cytotoxic. In fact some natural processes in the immune system utilise ROS as a way of destroying unwanted cells. These species have a short lifetime (<0.04 ms) and act in a short radius (<0.04 mm) from their point of origin. The destruction of cells leads to a necrotic-like area of tissue which eventually sloughs away or is resorbed. The remaining tissue heals naturally, usually without scarring. There is no tissue heating and connective tissue such as collagen and elastin are unaffected. This results in less risk to the underlying structures compared to thermal laser techniques, surgery or external beam radiotherapy. More detailed research has shown that PDT induces apoptosis (non-inflammatory cell death), and the resulting necrosis (inflammatory cell lysis) seen is due to the mass of dying cells which are not cleared away by the immune system.

There are several advantages of PDT. It offers non-invasive, low toxicity treatments which can be targeted by the light activation. The target cells cannot develop resistance to the cytotoxic species (ROS). Following treatment, little tissue scarring exists. However, currently available photosensitizing drugs are not very selective for the target cells only which induces collateral damage to the surrounding tissue in many situations this lack of selectivity leads to unacceptable damage to normal tissues with inflammation, pain, delayed healing and scarring with bad cosmetic and functional outcome, e.g. Photofrin™ in oesophageal or bladder cancer. Because systemic applied photosensitizer drugs often “piggy-back” on blood proteins with decreased renal clearance, they persist longer than desired in the system leaving the patient photosensitive for 2 weeks in the best of cases.

Currently, an approach to link photosensitizer drugs to targeting elements is the direct conjugation of derivatized photosensitizers to monoclonal antibodies. Whole antibodies have a high molecular weight in a range of 150 KDa, resulting in very large photo-immunoconjugates with unfavourable pharmacokinetics, such as poor tumour tissue vs. healthy tissue ratios (2:1) which reduces the therapeutic concentrations in tumour tissue and makes the therapy less efficient. Current literature suggest that photosensitizer drugs linked to residues on a monoclonal antibody can have a detrimental effect on each other, with quenching effects occurring due to poor spectroscopic properties. In addition to this, it has been shown that poor, and unreliable, loading of photosensitizer onto the antibody is seen with ratios of 4:1 being typical before the antibody aggregates or loses function.

In addition, antibodies are difficult to synthesize and, because of their large structure, are too large to enter through the skin barrier of a patient. Therefore, photosensitizer drugs having antibodies are not very useful for topical applications.

Others have tried to circumvent these problems by attempting to link photosensitizer drugs to designated ‘carriers’ such as branched carbohydrate or polyethylene glycol chains and polylysine chains. These approaches all require additional conjugation steps as the ligand-carriers cannot be made entirely recombinantly. Using such polymers may also have problems such as proteolytic instability in vivo. It is known that when photosensitizers are attached in this way, they self-quench, destroying their photophysical properties and rely on degradation in lysozymes to ‘de-quench’ before they can become active photosensitizers. Therefore, higher coupling ratios can be achieved, up to 10:1, but only with lower phototoxicity and lower singlet oxygen yield than that obtained with free (un-coupled) photosensitizer. Studies showed that the photosensitising activity of pheophorbides when covalently linked in large numbers around the periphery of a dendrimer were dramatically reduced. This is a result of energy transfer processes, mainly Forster energy transfer from dye to dye. Forster transfer is distance dependant and drops off rapidly with distance. The interaction of dye molecules leads to changes in the absorption spectrum, reduced fluorescence lifetimes and singlet oxygen quantum yields. Fusion proteins combining an antibody fragment with a protein carrier molecule have also been reported. All above methods were demonstrated on non-melanoma cancer tissues and very limited attempt has been made for melanoma cancer treatment. One possibility we found was to bind the photosensitizer to Napamide, an octapeptide derivative of α-melanocyte-stimulating hormone (α-MSH) that contains a chelator (DOTA) for radiometals and thus transport and accumulate radioactivity in melanoma cells and melanoma tumours of experimental animals. The DOTA-MSH conjugate specifically binds to MC1R which is overexpressed in malignant melanoma cells (and also in melanocytes). The ratio of radiometal concentration (e.g. ¹¹¹indium, ^{67/68}gallium or ⁹⁰yttrium) in tumour tissue vs. healthy tissue in experimental animals was very favorable so that the principle of this targeting concept was judged to be ideal for a novel to control melanogenesis and target melanomanotic skin lesions by photodynamic therapy (not using radioligands).

In conclusion, current photodynamic treatment strategies are efficient and used in clinical settings, but they are not targeted enough with tremendous inflammation, necrosis, pain and delayed healing. Therefore there is an urgent need for novel drugs or active compounds with improved selectivity for photodynamic therapy to treat in a targeted way various cancerous diseases and/or infections, in our case particularly treatment of melanotic lesions, such as Lentigo Maligna Melanomas, melanotic praecanserosis, Lentigenes and also for treatment of postinflammatory hyperpigmentations or for skin whitening.

The listing or discussion of an apparently prior-published document in this specification should not necessarily be taken as an acknowledgement that the document is part of the state of the art or is common general knowledge.

Any document referred to herein is hereby incorporated by reference in its entirety.

In an aspect of the present invention, there is provided a composition comprising a photosensitising agent covalently coupled to a ligand, wherein the ligand selectively binds to a targeted receptor. More particularly, the ligand selectively binds to a targeted receptor that is present in cells within an epidermal, a dermal or a subcutaneous tissue layer. The ligand may be any isolated peptide molecule (for example, 20, 15, 10 or less amino acids), protein (polypeptide), lipid, carbohydrate, alkaloid or combination thereof that selectively binds to a target cell, tissue or organism. In an embodiment, for topical application of the composition (which will be described in detail below), it is preferred to have ligands that are smaller molecules (e.g. alkaloids) and peptides and also smaller photosensitizers to improve and allow transcutaneous absorption and less enzymatic metabolism in the epidermis. Preferably, the photosensitising agent or photosensitiser produces ROS at various wavelengths of light. In an embodiment, the ratio of photosensitising agent to ligand is 1:1. The conjugation may be a covalent one.

By “epidermal layer”, it is meant to mean the epidermis of an organism (for example, a human person), which is a stratified squamous epithelium, composed of proliferating basal and differentiated suprabasal keratinocytes which acts as the body's major barrier against an inhospitable environment, by preventing pathogens from entering, making the skin a natural barrier to infection. The “dermal layer” is the layer of skin between the epidermis (with which it makes up the cutis) and subcutaneous tissues, that consists of connective tissue and cushions the body from stress and strain.

The terms “peptide” or “amino acid sequence” refer to an oligopeptide, peptide, polypeptide or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 8 amino acids and most preferably less than 10 amino acids. To be active, any polypeptide must have sufficient length to display biological and/or immunological activity but small enough to overcome the skin barrier of a patient in need of the compound for therapy. However, it will not be limited to peptides, the ligands could also be carbohydrates, lipids or alkaloids binding specifically to structures or receptors at the target cell or target organism (e.g. bacteria, fungi, virus, parasites).

By “ligand”, it is meant to include any molecule that could target a receptor of a diseased cell with high specificity and has functionality for covalent conjugation to the photosensitizing agent. The ligand may be any peptide, antibody, lipid, Alkaloid or carbohydrate or combination thereof. Such ligand may be any marker associated with a disease. Preferably, the ligand is an antagonist of the targeted receptor. In our model case, the targeted melanocortin 1 receptor is expressed in a melanocyte. The ligand may be monovalent or polyvalent.

In an embodiment, the ligand is Ac-Nle-Asp-His-D-Phe-Arg-Trp-Gly-Lys-NH₂.

Preferably, the composition further comprises a linker molecule for conjugating the photosensitising agent and ligand. The linker molecule may be any one selected from the group comprising: a polyethylene glycol unit, an amino acid derivative, and a bromo acid derivative. In an embodiment, the linker molecule is 4-bromomethylbenzoic acid.

By “photosensitizing agent”, it is meant to include any agent or compound useful in PDT. Such agents, when exposed to a specific wavelength of light, produce a form of oxygen that kills nearby cells. The photosensitizing agent may be porphyrin, protoporfin IX, verteporfin, HPPH, temoporfin, methylene blue. Preferably, the photosensitizing agent of the present invention is activated by light having a wavelength of between 400 nm to 700 nm. Still more preferably, the photosensitizing agent in the present invention is activated at 627 nm and 660 nm for the selective killing of melanotic cell with minimum killing of keratinocytes.

In an embodiment, the photosensitising is methylene blue. Alternatively, verteporfin, protoporfin IX, HPPH, temoporfin, photofrin, hematoporphyrin, Talaporfin, benzopophyrin derivative monoacid, 5-aminileuvolinic acid, metallophthalocyanine, zinc tetrasulfophthalocyanine, bacteriochlorins, chlorine derivative, or porphyrin derivatives may be used.

Preferably, the functional and photo-physical properties of the photosensitising agent and ligand are substantially unaltered in the coupled form in comparison to the properties when in an uncoupled form.

In another aspect of the present invention, there is provided the use of the compound in the diagnosis and/or treatment and/or prevention of a disease requiring the destruction of a target cell.

The disease may be anything benign, malign, infectious (caused by any infectious agents, for example any bacteria, virus or microbe or parasites) or inflammatory in nature. Preferably, the disease involves any tissue layers that are accessible by light (for example, skin, mucous membranes, cavities or the like) and/or endoscopic in nature. Preferably, the disease to be treated is cancer, infection, but can also include cosmetic applications. For topical application, preferably skin cancer. Such cancers may be include hyperplasia. Alternatively, the composition of the present invention may be useful to treat other skin conditions such as keloids.

Still alternatively, the composition may be used in cosmetics, for example, in whitening skin.

Preferably, diagnosis of disease is conducted by visualisation of the photosensitising agent.

The compound has to be administered to a patient prior to light exposure.

In yet another embodiment of the present invention, there is provided a pharmaceutical composition comprising the compound and a pharmaceutically-acceptable carrier, excipient or diluent.

Preferably, the formulation is a unit dosage containing a daily dose or unit, daily sub-dose or an appropriate fraction thereof, of the active ingredient.

The compounds of the invention may normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and age of patient to be treated, as well as the route of administration, the compositions may be administered at varying doses and formulations.

In human therapy, the compounds of the invention can be administered alone but will generally be administered in admixture with a suitable pharmaceutical excipient diluent or formulation or carrier selected with regard to the intended route of administration and standard pharmaceutical practice.

The compounds of the invention can be administered orally (via tablets and capsules) or parenterally, for example, intravenously, intra-arterially, intraperitoneal, intrathecal, intraventricular, intrastemally, intracranially, intra-muscularly or subcutaneously, or they may be administered by infusion techniques. For these kind of applications they should be used in the form of a sterile solution which may contain other necessary additives. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds has to be evaluated by further clinical studies. Thus, for example, the tablets or capsules of the compound of the invention may contain a dose of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

Alternatively, the compounds of the invention can be administered in the form of a suppository or pessary, or they may be applied topically in the form of a lotion, solution, cream, ointment or dusting powder. The compounds of the invention may also be transdermal administered, for example, by the use of a skin patch. They may also be administered by the ocular route, particularly for treating diseases of the eye. For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Generally, for skin lesions topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

In another aspect of the present invention, there is provided a method of making a compound comprising a photosensitising agent coupled to a ligand, the method comprising (a) providing a photosensitising agent; (b) providing a ligand, the ligand selectively binds to a targeted receptor present in cells within an epidermal, a dermal or a subcutaneous tissue layer; and (c) conjugating the photosensitising agent and ligand.

Preferably, a linker molecule is used for conjugating the photosensitising agent and ligand. More preferably, a linker molecule is a polyethylene glycol unit, amino acid derivative, bromo acid derivatives. In an embodiment, the linker molecule may be 4-bromomethylbenzoic acid or is made from 4-bromomethylbenzoic acid.

Advantageously, we have developed an elegant combinatorial strategy by covalent conjugation of first generation photosensitizers to a specific MC1 receptor peptide antagonist for the targeted delivery to melanotic cells and sequential precise LED light dosage to induce specific phototoxicity on melanotic cells with more MC1 receptor in the membrane than the surrounding cells. When administered to a patient, the provision of a light source that penetrates deep enough and damages specifically the targeted cells with minimal collateral damage to the surrounding tissues. The specificity will be increased by the very specific and well defined and localized LED light irradiation at near infrared wavelength (627 nm, 660 nm). In addition, the near infrared wavelengths allow deep skin penetration into the subcutaneous tissue. This accurate tandem therapy using a lock and key paradigm with preferential accumulation of photosensitizers in melanocytes rather than keratinocytes and fibroblasts lead potentially to extensive decline of collateral damage.

In order that the present invention may be fully understood and readily put into practical effect, there shall now be described by way of non-limitative examples only preferred embodiments of the present invention, the description being with reference to the accompanying illustrative figures.

In the Figures:

Scheme 1. Illustration of targeted delivery of photosensitizer prompted by MC1 receptor specific antagonist peptide and irradiation of near IR light.

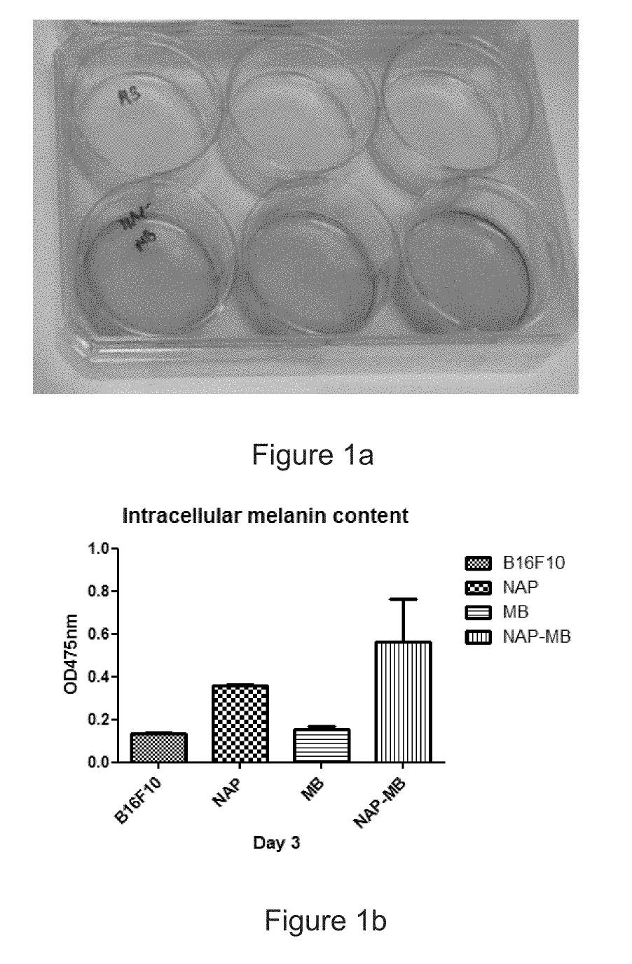

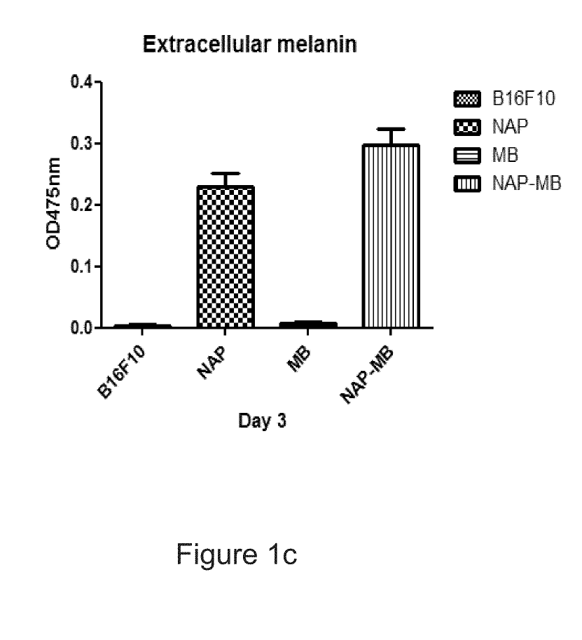

FIG. 1. a) Visual color change owing to melanin production was shown in triplicate upon addition of MB and NAP-MB at 1 μM concentrations. Melanin production assays b) intracellular, c) extracellular by incubating murine melanoma B16F10 cell line (45,000 cells/well) with MB (1 μM) and NAP-MB (1 μM) measuring absorbance at 475 nm.

Scheme 2. Synthesis of NAP-MB from 4-bromomethylbenzoic acid.

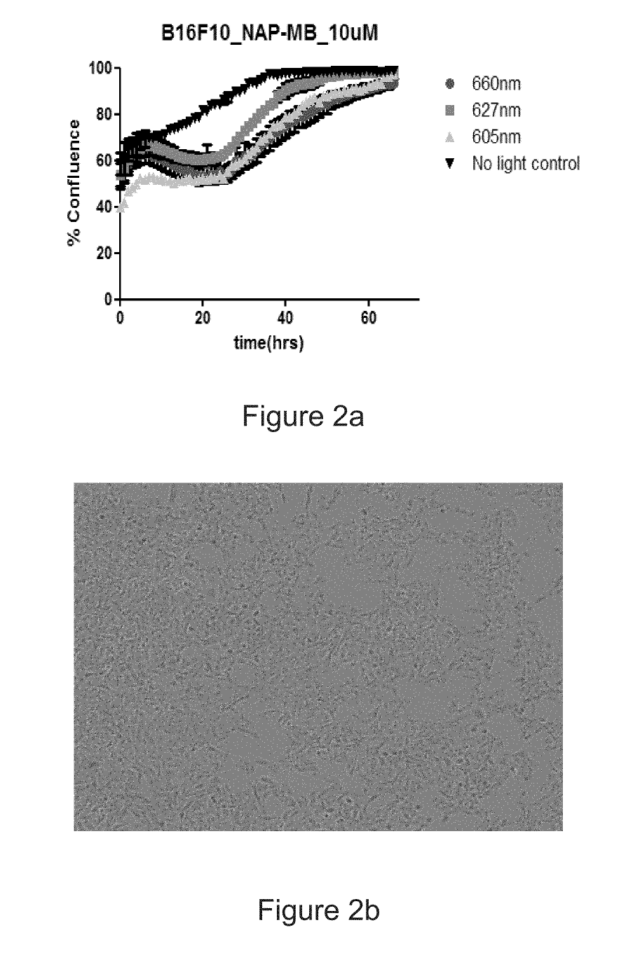

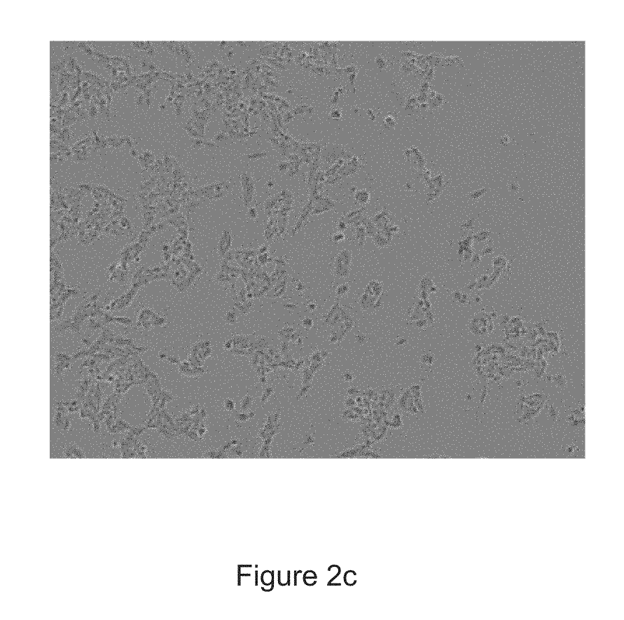

FIG. 2. a) Cytotoxicity effect of NAP-MB (10 uM) in B16 F10 cells at 605 nm (orange), 627 nm (red), 660 nm (brown) and with no light (black) irradiation. Cell growth image of B16F10 incubated with NAP-MB after 24 h with no light (b) and at 660 nm (c).

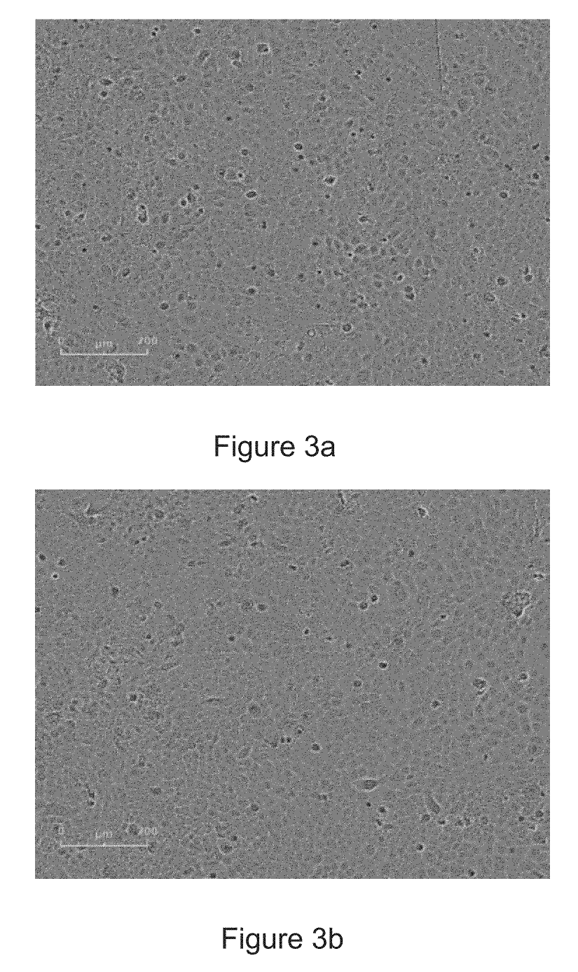

FIG. 3. Cell proliferation image of NTERT-1 incubated with NAP-MB after 24 h with (a) no light, at (b) 660 nm.

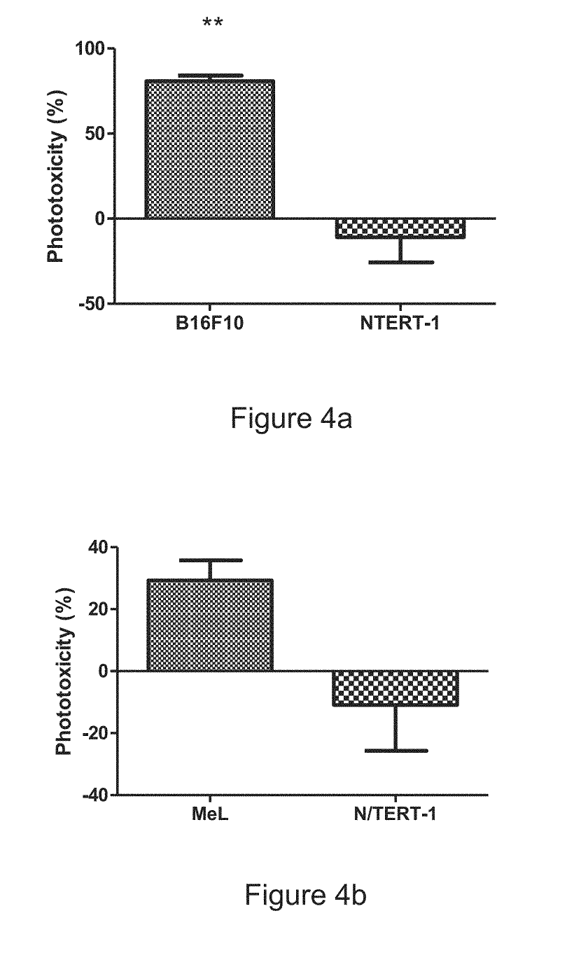

FIG. 4. Phototoxicity (%) of NAP-MB (1 μM) with B16-F10, MeL and N/TERT-1 measured by SRB colorimetric assay (Significance: *p≤0.05). The data represents mean value±SEM of three independent experiments with triplicates.

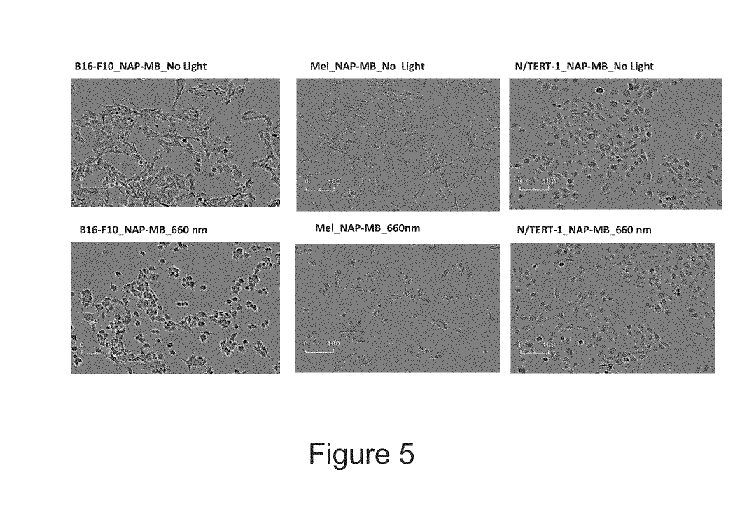

FIG. 5. Incucyte cell growth images for mouse melanoma cells, B16-F10, primary human melanocytes, MeL and human keratinocytes, N/TERT-1 after 24 h under no light and 660 nm light conditions. Images are representative images for single experiment performed thrice independently.

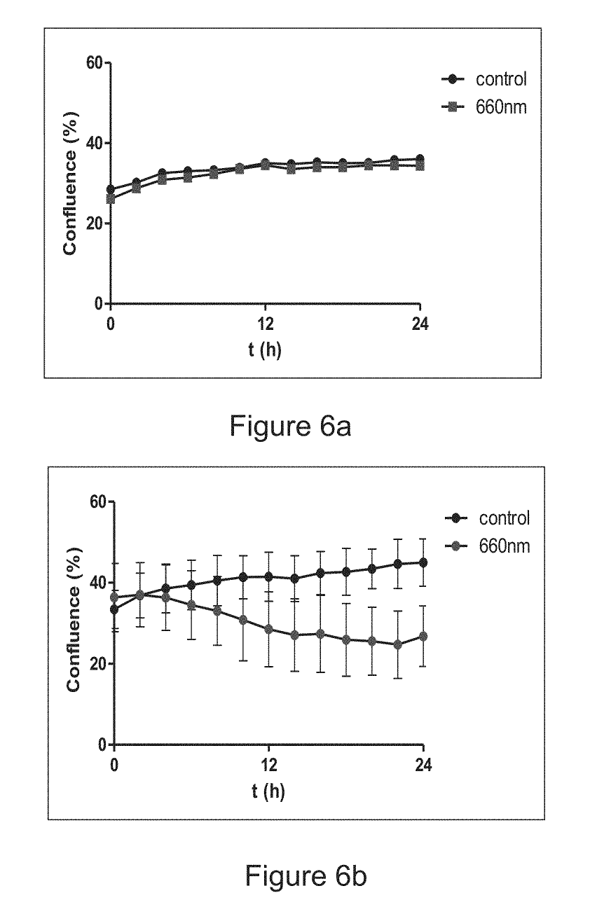

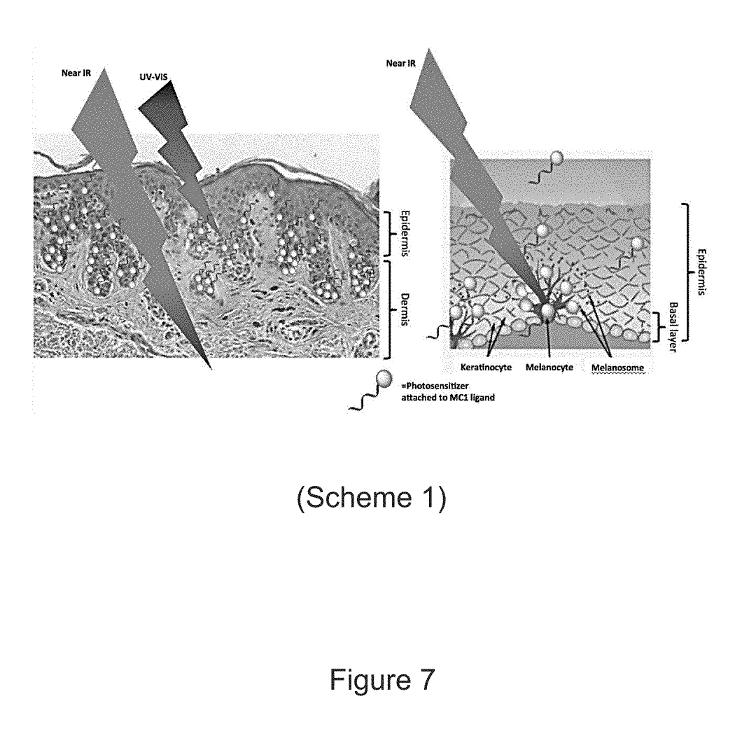

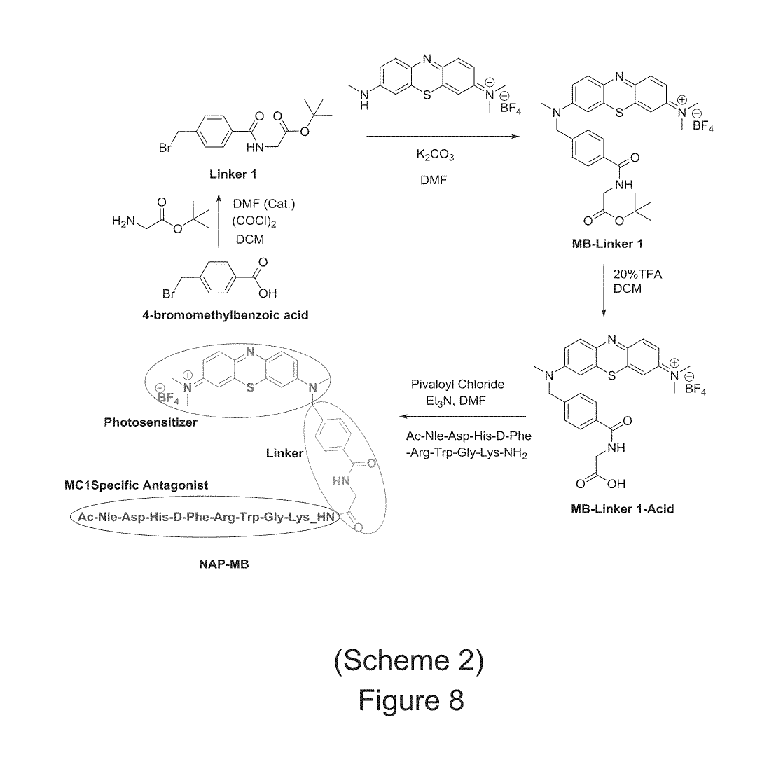

FIG. 6. Growth proliferation curves of primary human melanocytes a) with 660 nm light alone b) treated with NAP-MB (1 μM) and 660 nm light for 24 h.

### EXAMPLE

Material and Methods:

Chemical and anhydrous solvents were obtained from Sigma Aldrich, and were used without further purification. Spectroscopic grade solvents were purchased from Sigma Aldrich. Peptide sequences were purchased from Nova-Biochem. Anhydrous solvents were transferred using oven-dried syringe. The flash column was used to purify all the synthetic intermediates. The purification of peptides was performed by preparative reverse phase HPLC using Jupiter C12 Proteos 90 Å RP-HPLC column employing a binary gradient of Solution A (0.1% TFA in H₂O) and solution B (0.1% TFA in acetonitrile). The purity of peptides was ascertained by analytical reverse phase HPLC using Jupiter C4 Proteos 90 Å RP-HPLC column employing a binary gradient of Solution A (0.1% TFA in H₂O) and solution B (0.1% TFA in acetonitrile). The HPLC purified fractions were freeze dried using Labonco lyophilizer at −60° C. and 0.01 mbar vacuum. The ¹H NMR spectra of all compounds were recorded on a Bruker 400 MHz NMR spectrometer. Mass spectra were analyzed by Water LC-micro spectrometer using H₂O/Acetonitrile (1:1, v:v). Absorption spectrum measurement was performed on a Varian technology international UV spectrometer using 96-well plates.

Cells seeding were done at densities of 70,000 cells and/or 45,000 cells per well in Dulbecco's modified Eagle Medium, DMEM media without phenol red on Nunc six well tissue culture plates. α-MSH was purchased from Sigma Aldrich and Abcam that binds to MC1R expressed on the melanoma cells and promotes production and release of melanin through c-AMP signaling pathway. (3-Isobutyl-1-methylxanthine) IBMX was purchased from Sigma Aldrich was used as an internal standard that increases c-AMP levels in cells leading to increased melanin production. Melanin absorbance assay were done using a Bruker Plate reader at optical density 475 nm.

1. Synthesis of NAP-MB (Scheme 2):

Synthesis of Linker 1:

To a stirred solution of 4-bromomethyl benzoic acid (100 mg, 0.465 mmol) in 4 mL dry DCM was added oxaloyl chloride (413 uL, 4.65 mmol) and 2 drops (Cat.) of DMF at room temperature. The reaction mixture was stirred for overnight and the solvent were evaporated using rotary evaporator and vacuum at room temperature. The yellow solid was dried under high vacuum for 3 h and dissolved in 4 mL dry DCM. DIPEA (243 uL, 1.395 mmol) was added to the above solution and followed by tert-butyl glycine (85.7 mg, 0.515 mmol). The reaction mixture was stirred for 6 h at room temperature. The solvent were evaporated and the crude reaction mixture was purified by silica gel column chromatography using methanol: DCM (1:99, v:v) to furnish linker derivative 1 (91 mg) in 60% yield.

¹H NMR (400 MHz, Chloroform-d) δ 7.88-7.77 (m, 2H), 7.54-7.43 (m, 2H), 4.63 (s, 2H), 4.16 (d, J=4.9 Hz, 2H), 1.53 (s, 9H).

MS (ESI+): m/z (%)=414.29 (100) [M-CO2]+, 415.28 (25) [M+H—CO2]+

Synthesis of MB-Linker 1:

To a stirred solution of Azure B (150 mg, 0.418 mmol) in anhydrous DMF (4 mL) were added K₂CO₃ (110 mg, 0.836 mmol) and linker 1 (137 mg, 0.418 mmol) under argon at 47° C. To the above mixture KI (60 mg, 0.418 mmol) were added. After 1.5 h of heating, linker 1 (137 mg, 0.418 mmol) was added. After 3 h of heating, additional amount of linker 1 (137 mg, 0.418 mmol) was added. After 5 h, the DMF solvent were evaporated at 43° C. using high vacuum and the crude reaction mass was subjected to silica gel column chromatography using MeOH:DCM (7:93, v:v) to furnish MB-linker 1 (50 mg) in 30% yield.

¹H NMR (400 MHz, Chloroform-d) δ 7.72 (d, J=8.4 Hz, 4H), 7.28-6.99 (m, 6H), 4.77 (s, 2H), 3.97 (d, J=5.1 Hz, 2H), 3.70-3.50 (m, 3H), 3.26 (s, 9H), 1.34 (s, 9H).

MS (ESI+): m/z (%)=414.29 (100) [M-CO2]+, 415.28 (25) [M+H—CO2]+

Synthesis of MB-Linker 1-Acid:

To a stirred solution of MB-linker 1 (24 mg, 0.0529 mmol) in 0.8 mL of DCM were added dropwise solution of trifluoroacetic acid (0.2 mL) at room temperature. The reaction mixture was stirred for 3 h and the consumption of tert-butyl ester monitored by analytical HPLC. The solvent were evaporated using rotary evaporator and vacuum. The crude reaction mass has taken further without purification.

Synthesis of NAP-MB:

To a stirred solution of MB-linker 1Acid (29 mg, 0.0529 mmol) in 1 mL of DMF was added DIPEA (27.6 μL, 0.1587 mmol) followed by pivaloyl chloride (7 μL, 0.0582 mmol) at room temperature. After 1.5 h, Peptide 2 (58 mg, 0.0529 mmol) was added drop wise in 1 mL of DMF. The reaction mixture was stirred for 3 h. The solvent were dried at high vacuum at 40° C. The crude reaction mass was purified by preparative HPLC using detector at 640 nm, 210 nm, 254 nm and following gradient of 0.1% TFA in H₂O as solvent A and 0.1% TFA in Acetonitrile as solvent B.

| Time (min) | Solvent B (%) | Flow rate (mL/min) |
|---|---|---|
| 0 | 20 | 5 |
| 2 | 30 | 5 |
| 20 | 85 | 5 |
| 22 | 85 | 5 |
| 23.5 | 20 | 5 |
| 25 | 20 | 5 |

The purity of freeze dried sample (8 mg) was 80% and was further purified using detector at 640 nm, 210 nm, 254 nm and following gradient.

| Time (min) | Solvent B (%) | Flow rate (mL/min) |
|---|---|---|
| 0 | 30 | 5 |
| 2 | 30 | 5 |
| 28 | 70 | 5 |
| 29.5 | 70 | 5 |
| 31 | 30 | 5 |
| 23 | 30 | 5 |

Column = Jupiter C4 Proteos 90 Å RP-HPLC
Gradient = Solution A (0.1% TFA in H₂O) and solution B (0.1% TFA in acetonitrile)

The purity of the fractions was ascertained by analytical HPLC using same gradient of solvents. The fractions of similar purity were combined and lyophilized. Weight of NAP-MB=3 mg; purity=100%.

2. Melanin Assay Procedure:

Murine melanoma B16F10 cells were seeded in Nunc six well tissue culture plates (45,000 and 70,000 cells per well) in DMEM media without addition of phenol red. Cells were stimulated by a positive control α-MSH (10 nM) or alternatively by using an internal standard IBMX (50 μM) for 18 h incubation of the cells. Photosensitizer (1 μM) or peptide conjugated photosensitizer (1 μM) were added at room temperature and incubated for 72 h at 37° C. The extracellular supernatants (A) were pipetted out from the cell pellets settled at the bottom of the well plates. Three concurrent measurements were taken employing 200 uL of supernatants at 475 nm (FIG. 1 b). Cells were detached with 0.02% EDTA solution and centrifuged at 2000 rpm for 3 minutes. Cell pellets were dissolved in 1M NaOH (2004), heated at 75° C. for 5 min to lyse the cells and cooled down to the room temperature. Three concurrent measurements were taken at 475 nm (FIG. 1c). Melanin production was tested using 1 μM of NAPamide, MB, NAP-MB were used as active drug components to demonstrate the binding MC1 receptor by measuring melanin production.

3. Cell Proliferation Assay:

Proliferation Experiments with Photosensitizers:

Murine melanoma B16F10 cells were seeded at 3000-4000 cells per well in 96 well black view plate Perkin Elmer plates in DMEM media without phenol, as phenol red may interfere in the absorption of light. After overnight incubation, media was aspirated from the wells and varying concentrations of photosensitizers were added to the cells. Cells were incubated for 4 hours in dark at 37° C., after 4 hours cells were washed twice with 1×PBS and 300 μL of media was added to the wells before placing the plates in Incucyte ZOOM Live cell imaging machine to capture the images after every hour. Percentage confluence was measured over a time period. Similar approach was taken for keratinocyte cell lines NTERT-1 except the starting confluence was 75-85%. This is to mimic the physiological situation in the body where keratinocytes are present in much larger numbers than melanocytes.

Proliferation Experiments with Photosensitizers and Light Irradiation:

B16F10 cells were seeded at 4000 and/or 4500 cells per well in DMEM media without phenol red overnight. Cells were incubated with the toxins at the desired concentrations for 4 hours in dark, cells were washed twice with 1×PBS and media was replaced. Cells were then irradiated at 605 nm, 627 nm and 660 nm for specific experiments using LED system. The power intensity was kept constant at 0.10 mW/cm2 for different duration of time. Cells were imaged after every hour for growth proliferation curves. Similar approach was taken for NTERT-1 cells except the starting confluence was 75-85%.

Cytotoxicity Study of MB and NAP-MB:

B16F10 cells were seeded at 3000 cells per well in a 96 well tissue culture plate. Cells were treated with 10 μM, 1 μM, 500 nM, 250 nM, 100 nM concentrations of MB for 4 hr and allowed to proliferate at 37° C. For the light experiments, B16F10 cells were seeded at density of 4500 cells per well in a 96 well plate, treated with NAP-MB (10 μM) for 4 hrs and exposed to red light at 0.10 mW/cm2 for 24 hrs continuously. Cells were imaged at every hour interval through incucyte (FIG. 2).

The following provides further data to show the specific targeting and destruction of melanoma with minimum collateral damage to normal cells by providing quantifiable cytotoxicity data of mouse melanoma cells B16-F10, primary human melanocytes, MeL and human skin keratinocytes N/TERT-1.

Proliferation Experiments with Synthetic Peptide-Photosensitizer Construct NAP-MB and Light Irradiation:

Mouse melanoma cells B16-F10, human skin keratinocytes N-TERT-1 and primary human melanocytes MeL at cell densities of 4000 cells/well, 6000 cells/well and 3500 cells/well respectively were seeded to achieve similar starting confluence in 96-black Perkin-Elmer well plate in DMEM medium without phenol red (for B16F10 and MeL) and KSFM media (for N/TERT-1 cells) and kept for overnight at 37° C. The cells were incubated with NAP-MB at 1 μM concentration in the dark for 4 h and then the cells were irradiated at 660 nm wavelength light using Incucyte-LED system. The light intensity was kept constant at 0.10 mW/cm² for 24 hours. Percentage confluence was measured over a time period. The images obtained after 24 h were used for comparative analysis of combinatorial effect of light and NAP-MB on cell morphologies.

Sulphorhodamine (SRB) Phototoxicity Assay:

Mouse melanoma B16-F10 cells, human keratinocytes N/TERT-1 and primary human melanocytes MeL, were seeded at above mentioned densities to achieve similar confluence after overnight incubation to perform SRB cytotoxicity assay. Cells were incubated with 1 μM of NAP-MB for 4 h, and left in the medium with 0.10 mW/cm² of 660 nm light irradiation for 24 h. After the end of the experiment, cold 10% trichloroacetic acid (100 μL) was added to the wells. Upon one hour incubation at 4° C., the cells were washed five times with water and air dried. Further, SRB in 1% acetic acid (0.4%, 100 μL) was added to each well. After 30 min of incubation at room temperature, cells were washed thrice with 1% acetic acid and air dried. 10 mM Tris-base (200 μL) was added to solubilize the dye with gentle agitation. Optical density was measured at 510 nm wavelength. The percentage of phototoxicity was calculated using the following equation:

$$Phototoxicity ( \% ) = 1 - \frac{( {OD}_{Treatment} - {OD}_{Blank} )}{( {OD}_{Control} - {OD}_{Blank} )}$$

Where—

OD_{Treatment}=Optical density measured for cells treated with peptide-photosensitizer NAP-MB and light at 660 nm.

OD_{Blank}=Optical density measured for media, DMEM or KSFM

OD_{control}=Optical density measured for cells treated with peptide-photosensitizer NAP-MB.

Results:

Phototoxicity Assay with NAP-MB (SRB Assay)

Most of the effective photosensitizers available for therapeutic applications are ideally known to have renal clearance at least 24 h in real physiological conditions. To increase systemic presence, NAP-MB was incubated along with light irradiation for 24 h and sulforhodamine B (SRB) cytotoxic colorimetric assay was performed to quantify the amount of phototoxicity to the cells at 1 μM NAP-MB using melanoma cells (B16-F10), human keratinocyte cells (N/TERT-1) and primary human melanocytes (MeL).

Mouse melanoma cells, B16-F10 owing to abundant presence of MC1R were significantly affected by the conjugated NAP-MB photosensitizer as compared to N/TERT-1 cells (FIG. 4a). This establishes the specificity with which NAP-MB targets the MC1R on melanoma cells as a quantifiable proof of the specific targeting and toxic nature of NAP-MB to melanoma cells. Primary human melanocytes were also tested and though some toxicity was observed but not to the levels of B16-F10 cells owing to the less than 10 fold MC1 receptors on melanocytes than melanoma cells (FIG. 4b). Melanocytes were not affected by light stimulation alone for continuously 24 hours as can be visualized in the graph (FIG. 6a). When incubated with NAP-MB and irradiated with 660 nm light for continuous 24 hours reduced proliferation was noticed in cells treated with NAP-MB and light as compared to only NAP-MB treated cells not exposed to light (FIG. 6b).

Appreciable cell morphology difference was found after 24 h light incubation with NAP-MB with B16-F10 melanoma cells and melanocytes. Cells clearly showed unhealthy morphologies compared to non-light exposed controls. N/TERT-1 keratinocytes proliferated and retained normal cell morphology even with the treatment of photosensitizer and light (FIG. 5).

Thus, both quantitative and qualitative assays demonstrate extensive collateral damage reduction by killing melanoma cells and melanocytes specifically over human keratinocytes thereby achieving our objective of specific targeting of MC1R positive cells.

### DISCUSSION

The efficacy of PDT depends on three components:
1) the light source that emits specific wavelengths and doses. The penetration of the light into tissue depends widely from the wavelengths. In skin, the UV and blue light penetrate much less than the red and infrared spectra (see Scheme 1);
2) the photosensitizer that releases with a given light quality Reactive Oxygen Species (ROS) that leads to cellular necrosis; and
3) the target tissue and cells that is damaged by the ROS. The efficacy of PDT on the target tissue depends on the absorption of the photosensitizer into the tissue and the incorporation of the photosensitizer into the cells.

The proposed PDT has a high potential to target specific tissue, cells and infectious organisms and the precision and efficacy depends on light source and the photosensitizers. Therefore, PDT is used and has a high future potential in dermatology and many other medical specialties such as urology, gastroenterology to treat superficial epithelial and pigmentary cancers, infections or even for cosmetic applications (e.g. skin whitening, coup rose in Rosacea, Acne). It is already approved for treatment of non-melanoma skin cancers such as superficial basal cell carcinoma, Bowen's disease and has also shown efficacy in treatment of common, recalcitrant HPV infections, Leishmaniasis, Acne, Rosacea and others with the porphyrin precursor delta-aminolaevulinic acid. However, all of photosensitizers used until now in PDT are non-specifically accumulated in hyperproliferating tissue (e.g. epithelial cancer cells) with a considerable collateral damage and tremendous pain issues. Our approach targets specifically the MC-1 receptor (as an example) that is expressed in higher quantities on melanotic cells using MC1 receptor antagonist (NAPAmide) attached to photosensitizers such as methylene blue, HPPH, verteporfin and precise light wavelengths generated by a suitable LED device as light source for targeted therapy can take photodynamic therapy a step further.

We tried to address all the concerns regarding photosensitizer such as low solubility in experimental media, bulky size, high molecular weight, hydrophobicity, and more importantly, need of high reactive oxygen species (ROS) quantum yield.

After literature screening, small size and molecular weight, we identified a positively charge bearing methylene blue, a photosensitizer that has improved solubility and has moderate to good ROS quantum yield, was taken for further cytotoxicity and cell proliferation study. The methylene blue (MB) was covalently attached to MC1 receptor specific peptide antagonist Ac-Nle-Asp-His-D-Phe-Arg-Trp-Gly-Lys-NH2 (NAP-NH2) using a linker as shown in Scheme 2.

Substantial production of extracellular as well as intracellular melanin indicates the targeting and binding nature of these peptides and peptide conjugated photosensitizers to the MC1 receptor present in both murine melanoma B16F10 cell line and human mid pigmented FM 55 cell lines. Whereas, negligible or less melanin production was observed with photosensitizer alone (FIG. 1b,c). The color change due to huge melanin generation was visualized by naked eye (FIG. 1a).

Incubating murine melanoma cell line B16F10 with 10 uM of MB and NAP-MB in dark, no cytotoxicity was observed. Upon irradiating the B16F10 cells continuously for 24 hrs at 0.10 mW/cm² energy intensity, proliferation defect was visible at 605 nm, 627 nm and 660 nm wavelengths of light with maximum damage at 660 nm (FIG. 2). The cell proliferation was retained after switching off the light source.

At this juncture, we were interested to see the effect of light at these wavelengths on keratinocyte NTERT-1 cells, which express much less MC1 receptor to explain minimum collateral damage. Less cell toxicity was observed for NTERT-1 keratinocyte cell type even after 24 hours of light exposure at 660 nm (FIG. 3) leading us to believe that MB is a better photosensitizer for targeted photodynamic therapy for the precise treatment of melanoma melanogenesis with minimal collateral damage. We need to repeat our experiments with other skin cell types to identify the extent of collateral damage and toxicity and so far NAP-MB is our promising lead candidate.

In the present invention, the specific target site lies within the skin cells (single type cell) i.e. melanotic cells for controlling melanogenesis making minimum or no damage to keratinocyte cells. The preferential achievement of specificity within one type of cell may be a challenging task. The complexity increases with the presence of abnormal melanotic cell in upper layer of the skin or in the lower layer of the skin. This could be solved by replacing methylene blue by another photosensitizer such as verteporfin, temoporfin, protofrin, protoporhyrin IX, HPPH or future novel photosensitizers with the required wavelength for activation individual for each compound. Changing various photosensitizers of different wavelengths without changing the peptide can address the melanoma malignancies from superficial to deeper (transcutaneous) melanoma or use in cosmetics for skin whitening. By manipulating intensity of lights used, time of irradiation, and amount of reactive oxygen species generated, the present invention can treat small to big melanoma tumours of different age groups and different skin types. The present compounds may be used in treatment strategies (including varying different intensities of radiation) that address different delicate body locations (for example, face) for melanogenesis.

Unlike systemic tumours, the site of tumour is present in the different skin layers (from epidermis to subcutaneous tissue). Therefore, the sensitizer can be administered through transdermal delivery (with or without occlusion) as cream, ointment, patch or by microneedles or by intralesional injections, less systemic approaches. The light source comes usually from outside by a lamp or even endoscopic for internal organs or maybe could be implanted into the lesion with battery and remote control.

We developed an elegant combinatorial technology by covalent conjugation of first generation photosensitizers to the MC1 receptor specific peptide antagonist for the targeted delivery to melanotic cell and irradiation of sequential LED light dosage at precise wavelength kills melanotic cells in melanoma was achieved successfully with minimum collateral damage.

This technology of site-specific chemical accumulation and precise and localized light delivery generated by LED for different penetration level has the ability to execute targeted therapy in microscopic and nanoscopic environment is demonstrated.

This technology has the potential to treat benign hyperpigmentation and also large surface superficial malign melanotic lesions, such as Lentigo Maligna Melanomas (LMM) with a reach up to the dermis (dependent on the transcutaneous absorption of the photosensitizer). The near infrared wavelengths can reach lower dermal compartments. This presents a first-in-class strategy that uses photosensitizer conjugated to ligand and light wavelength of LED. This gives us a platform to play with various therapeutic conditions such as:
a. targeted concentration and accumulation of novel chemical ligation of photosensitizer with specific ligand that binds to MC1 receptor (particularly expressed in melanotic cells); The preferred accumulation ratio between melanotic tissue and healthy peripheral tissue is less than 4:1.
b. various wavelengths and duration of irradiation of same photosensitizer to generate different extent of ROS and also depths into the tissue;
c. the photosensitizers can be changed; and other photosensitizers such as verteporfin, protoporfin IX, HPPH, temoporfin, photofrin, hematoporphyrin, Talaporfin, benzopophyrin derivative monoacid, 5-aminileuvolinic acid, metallophthalocyanine, zinc tetrasulfophthalocyanine, bacteriochlorins, chlorine derivative, porphyrin derivatives may be used.
d. multi-hit treatment of malign lesions also possible by ligation of the photosensitizers to other, specific ligands or antibodies to other targets (e.g. blood vessels, specific tumour markers),

to address different skin type (Asian, African etc.) disorder, different penetration level of disorder (superficial, cutaneous, etc), different age of patient, and different stage of diseases (early or late stage) the novel ligated photosensitizers have to be combined with the right formulation or even delivery by microneedles.

Whilst there has been described in the foregoing description preferred embodiments of the present invention, it will be understood by those skilled in the technology concerned that many variations or modifications in details of design or construction may be made without departing from the present invention.

1. A composition comprising a photosensitising agent coupled to a ligand, wherein the ligand selectively binds to a targeted receptor present in cells within an epidermal, a dermal or a subcutaneous tissue layer.

2. The composition according to claim 1, wherein the ratio of photosensitising agent to ligand is 1:1.

3. The composition according to claim 1, wherein the ligand is an antagonist of the targeted receptor.

4. (canceled)

5. The composition according to claim 1, wherein the targeted receptor is melanocortin 1 receptor.

6. (canceled)

7. The composition according to claim 1, wherein the ligand is Ac-Nle-Asp-His-D-Phe-Arg-Trp-Gly-Lys-NH₂.

8. The composition according to claim 1, further comprising a linker molecule for conjugating the photosensitising agent and ligand, wherein the linker molecule is selected from the group comprising: a polyethylene glycol unit, an amino acid derivative, and a bromo acid derivative.

9. (canceled)

10. The composition according to claim 8, wherein the linker molecule is 4-bromomethylbenzoic acid.

11. (canceled)

12. The composition according to claim 1, wherein the photosensitising agent is any one selected from the group comprising: methylene blue, verteporfin, protoporfin IX, HPPH, temoporfin, photofrin, hematoporphyrin, Talaporfin, benzopophyrin derivative monoacid, 5-aminileuvolinic acid, metallophthalocyanine, zinc tetrasulfophthalocyanine, bacteriochlorins, chlorine derivative, or porphyrin derivatives.

13. A method of making a compound comprising a photosensitising agent coupled to a ligand, the method comprising:
(a) providing a photosensitising agent;
(b) providing a ligand, the ligand selectively binds to a targeted receptor present in cells within an epidermal, a dermal or a subcutaneous tissue layer; and
(c) conjugating the photosensitising agent and ligand.

14. The method according to claim 13, further providing a linker molecule for conjugating the photosensitising agent and ligand, wherein the linker molecule is any one selected from the group comprising: a polyethylene glycol unit, an amino acid derivative, and a bromo acid derivative.

15. (canceled)

16. The method according to claim 13, wherein the linker molecule is 4-bromomethylbenzoic acid.

17. A compound comprising the composition of claim 1, or obtainable by the method according to claim 13.

18. The compound according to claim 17, wherein the functional and physical properties of the photosensitising agent and ligand are substantially unaltered in the coupled form in comparison to the properties when in an uncoupled form.

19. (canceled)

20. (canceled)

21. A compound according to claim 17, for use in the diagnosis and/or treatment and/or prevention of a disease requiring the destruction of a target cell.

22. (canceled)

23. The compound of claim 21, wherein the disease to be treated is cancer of the skin.

24. The compound of claim 21, wherein the treatment includes skin whitening.

25. The compound of claim 21, wherein diagnosis of disease is conducted by visualisation of the photosensitising agent.

26. The compound of claim 21, wherein the compound is administered to a patient prior to light exposure.

27. The compound of claim 21, wherein the target cell is a cell within an epidermal, a dermal or a subcutaneous tissue layer.

28. A pharmaceutical composition comprising a composition according to claim 1 or a compound according to claim 12 and a pharmaceutically-acceptable carrier, excipient or diluent.

29. A pharmaceutical composition comprising the composition of claim 1 and a pharmaceutically-acceptable carrier, excipient or diluent.
